(19) United States
(12) Patent Application Publication
(10) Pub. No.: US 2022/0323536 A1
(43) Pub. Date: Oct. 13, 2022

(54) USE OF BETA-L-ASPARTYL-L-ARGININE ON SENESCENT SKIN

(71) Applicant: SYMRISE AG, Holzminden (DE)

(72) Inventors: Dominik STUHLMANN, Holzminden (DE); Ann-Christin WESELOH, Polle (DE); Imke MEYER, Bodenwerder (DE)

(21) Appl. No.: 17/639,081

(22) PCT Filed: Aug. 28, 2020
(86) PCT No.: PCT/EP2020/074130

§ 371 Date: Feb. 28, 2022

(30) Foreign Application Priority Data
Aug. 30, 2019 (EP) PCT/EP2019/073272

(51) Int. Cl. A61K 38/05; A61K 9/00; A61K 8/64; A61Q 19/08; A61P 39/06

(52) U.S. Cl. CPC A61K 38/05; A61K 9/0014; A61K 8/64; A61Q 19/08; A61P 39/06

(57) ABSTRACT

The present invention relates to β-L-aspartyl-L-arginine for use as an anti-inflammatory agent on the skin as well as the cosmetic use of β-L-aspartyl-L-arginine on mature skin. It particular, the β-L-aspartyl-L-arginine provides an antioxidative effect and/or improves mitochondrial function in senescent skin cells, in particular senescent fibroblasts, and/or restores firmness of the skin. Furthermore, the invention also provides a cosmetic method for the treatment of mature skin. The present invention also relates to β-L-aspartyl-L-arginine for use in a therapeutic method for the treatment of damaged mature skin. In particular, the β-L-aspartyl-L-arginine is provided for use in a therapeutic method to promote wound healing.

The present invention relates to β-L-aspartyl-L-arginine for use as an anti-inflammatory agent on the skin. Furthermore, the invention provides a the cosmetic use of β-L-aspartyl-L-arginine on mature skin. It particular, the β-L-aspartyl-L-arginine provides an antioxidative effect and/or improves mitochondrial function in senescent skin cells, in particular senescent fibroblasts, and/or restores firmness of the skin. Furthermore, the invention also provides a cosmetic method for the treatment of mature skin. The present invention also relates to β-L-aspartyl-L-arginine for use in a therapeutic method for the treatment of damaged mature skin. In particular, the β-L-aspartyl-L-arginine is provided for use in a therapeutic method to promote wound healing.

Mature skin is characterized by an increasing amount of senescent cells in the dermal layer (dermis), which no longer replicate and divide. Fibroblasts located within the dermal layer are its essential component. Their main function is to provide tensile strength and elasticity trough the production and secretion of the extracellular matrix components, including collagens and proelastin. Through aging, fibroblasts respond to damage and oxidative stress by entering a state of arrested growth and altered function called cellular senescence. Senescent fibroblasts secrete growth factors, cytokines, and degradative enzymes leading to a loss of skin elasticity, delay of wound healing, and changes of superficial morphology (Krtolica et al., PNAS Oct. 9, 2001, 98 (21), 12072-12077; Sorel and Caplan, J Cell Sci, 2004 Feb. 15; 117: 667-675; Ezure et al., IUBMB Journal, BioFactors 2019; 45:556-562; Sibilla et al., The Open Nutraceuticals Journal, 2015, 8:29-42).

Mitochondria are considered as the “powerhouse” of the cell. The main function of mitochondria is to assimilate nutrients (glucose) from the cell, break them down and convert them into energy (ATP). This energy is then used by the cell to carry out various biological functions. Mitochondrial dysfunction has been associated with many age-related disorders and photo-aging. Dysfunctional unhealthy mitochondria decrease energy production and increase oxidative stress (Krutmann and Schroeder, J Investig Dermatol Symp Proc, 2009; 14(1):44-9).

In functional mitochondria, oxygen uptake, ATP production and generation of reactive oxygen species (ROS) are tightly regulated to maintain the redox balance. Mitochondrial homeostasis impairment such as increased mitochondrial biogenesis, and decreased mitophagy together with decreased ATP production and increased ROS generation induce a senescence cell cycle arrest. Moreover, increased ROS levels can induce DNA damage which leads to a permanent cell cycle arrest. Senescent cells generate and secrete: growth factors, extracellular matrix degrading proteins and pro-inflammatory cytokines (SASP), which together with ROS not only stabilize senescence, but also induce paracrine senescence, which may contribute to the detrimental effects during aging (Korolchuk et al., EBioMedicine, 2017, 21: 7-13; Correira-Melo and Passos, Biochimica et Biophysica Acta, 2015, Volume 1847, Issue 11, 1373-1379). Moreover, the secretion of pro-inflammatory cytokines and growth factors by senescent cells (SASP=senescence associated secretory profile) causes chronic inflammation, which may ultimately lead to tissue damages.

Skin increasingly accumulates senescent cells during the natural aging process, which leads to a deterioration of its appearance and texture causing cosmetic issues such as loose skin and wrinkles. Moreover, injuries such as cuts and scratches tend to heal significantly slower in mature skin than in younger skin. In order to maintain the appearance and texture of younger skin, it is desirable to interfere with or ideally even reverse the effects of cellular senescence in the skin.

Dipeptides have been described for cosmetic use on the skin. WO 2017/162879 A1 relates to β-aspartyl dipeptides for skin care and cosmetic use. The dipeptides are shown to increase proliferation of basal keratinocytes in skin models and large amounts of the β-aspartyl dipeptides were found to improve skin elasticity on adult volunteers. However, there is no indication of the age of the volunteers in the in vivo tests and the amount of β-aspartyl dipeptides used is extremely large (5% aqueous solution) and therefore not economically feasible.

U.S. Pat. No. 5,478,560 A discloses that L-arginine-L-aspartic acid is able to retain moisture in the skin and accelerate the proliferation of cells.

It was an objective of the present invention to provide cosmetic treatment for mature skin, which interferes with or even reverses the effects that lead to an increase of senescent cells in the skin during the aging process and the associated cosmetic issues outlined above.

In particular, it was an object of the present invention to provide a cosmetic treatment for mature skin, which mitigates the effects of oxidative stress in skin cells, in particular the loss of mitochondrial function.

It was a further objective of the present invention to provide a cosmetic method of treating mature skin, which decreases the amount of senescent cells in the skin.

It was another objective of the present invention to provide an anti-inflammatory treatment for the skin, in particular a treatment that reduces or prevents the secretion of pro-inflammatory factors by senescent skin cells.

It was also an objective of the present invention to provide a treatment for damaged mature skin, in particular a treatment, which promotes wound healing.

The present invention relates to β-L-aspartyl-L-arginine for use as an anti-inflammatory agent on the skin, in particular to prevent or reduce secretion of one or more pro-inflammatory cytokines and/or growth factors by skin cells, preferably senescent skin cells.

It has surprisingly be found out in the context of the present invention, that β-L-aspartyl-L-arginine is capable of significantly reducing the secretion of pro-inflammatory cytokines and/or growth factors by skin cells, in particular senescent skin cell, when the dipeptide is applied topically to the skin.

Preferably, the pro-inflammatory cytokines and/or growth factors are selected from the group consisting of interleukin 6 (IL-6), interleukin 8 (IL-8), vascular endothelial growth factor (VEGF) and transforming growth factor (TGF).

In one embodiment the β-L-aspartyl-L-arginine is used in a method for the treatment and/or prevention of chronic inflammation of the skin, preferably chronic inflammation caused by the secretion of pro-inflammatory cytokines and/or growth factors by senescent skin cells.

The present invention also relates to a cosmetic use of β-L-aspartyl-L-arginine on mature skin for providing an antioxidative effect and/or improving mitochondrial function in senescent skin cells, in particular senescent fibroblasts, and/or restoring firmness of the skin.

It was found out in the context of the present invention, that β-L-aspartyl-L-arginine provides effects, which reduce the amount of senescent cells in mature skin. The dipeptide therefore allows cosmetic treatment with pronounced effects specifically on mature skin. A strong antioxidative effect was observed, which protects skin cells from oxidative damage and restores the redox balance to improve mitochondrial function. It is also envisioned that the observed effects may be applicable for hair care, in particular to prevent hair loss and hair greying due to a regenerative effect on hair follicles.

β-L-aspartyl-L-arginine can be obtained by chemical synthesis or by enzymatic degradation of cyanophycin obtained from blue-green algae. Blue-green algae synthesize cyanophycin granule peptides (CGP) for temporary nutrient and energy storage under rich environmental conditions. When the environmental conditions become harsh and nutrients are scarce, the algae use the energy and nutrients stored in the CGP biopolymers by splitting them into dipeptides and free amino acids and thus ensure their survival. Green chemistry and biotechnology (microbial fermentation) can be used to provide the marine-derived dipeptide. Advantageously, the product is genetically modified organism (GMO)-free and can be produced by white biotechnology in high purity. As a white, odorless powder, it is water soluble and stable at a pH from 5 to 9 at up to 50° C. and fulfills cosmetics safety and purity requirements. Methods for the preparation of dipeptides by hydrolysis of CGP using a peptidase (CGPase) are described in detail in WO 2009/150252 A1.

The term “mature skin” in the context of the present invention refers to skin, which comprises a significant amount of senescent cells. In particular, mature skin is the skin of an individual, which is at least 40, at least 45, at least 50, at least 55, at least 60, at least 65 or at least 70 years old.

In a preferred embodiment of the use described above, the mature skin is therefore the skin of an individual, which is at least 40, at least 45, at least 50, at least 55, at least 60, at least 65 or at least 70 years old.

An “antioxidative effect” in the context of the present invention is an effect, which inhibits oxidation reactions such as carbonylation of proteins in a cellular environment. Oxidation reactions can e.g. be induced by UV irradiation and can lead to the formation of free radicals. Free radicals produce chain reactions, which can do significant damage to the cell, in particular the DNA, initiating repair-mechanisms and oxidative stress reactions. As a result, the amount of reactive oxygen species (ROS) in the cell is increased.

As explained above, mitochondria assimilate nutrients from the cell, break them down and convert them into energy (ATP). In functional mitochondria, oxygen uptake, ATP production and generation of reactive oxygen species (ROS) are tightly regulated to maintain the redox balance. “Improved mitochondrial function” therefore refers to an effect, which improves or restores the energy production and the redox balance in the cell.

The skin has plastic and elastic components, which together describe the viscoelastic characteristic of the skin. When a force acts on the skin, the skin does not immediately return into its original state but remains initially in a state of slight deformation (hysteresis). In mature skin, the plastic momentum plays a larger role in the deformation and different skin areas show varying plasticity and elasticity. The method of measurement relies on suction. A vacuum is created in the gauge head of the cutometer, which sucks the skin into the measuring device. An optical arrangement, consisting of a light source and a light detector, measures the light intensity, which enters dependent on how much skin is sucked in. The resulting parameters are elasticity and skin firmness.

“Skin firmness” in the context of the present invention represents the resistance, which the skin creates against the suction by the vacuum. Elasticity, on the other hand, is the time, which the skin needs to return to its original state

In the context of the present invention, “senescent cells” are cells, which, due to damage and oxidative stress, have entered a state of arrested growth and altered function. In particular, senescent cell no longer divide but are still metabolically active. During aging, the number of senescent cells in tissues rises significantly. Further characteristics of senescent cells were already given above and are also described below.

Giant dysfunctional mitochondria in senescent cells are the result of mitochondrial ATP deficits compensation strategy and mitochondrial size can be used as a quantitative approach to evaluate senescent changes in the cell. Disorganization of chromatin in senescent cells leads to an enlarged nucleus. Therefore, nucleus/cell size ratio is another good marker of senescence (Hohn et al., Redox Biology, 2017, 13; 550-567; Chandra et al., Cell Reports, 2015; 10(4): 471-483).

Mitochondria contain mitochondrial DNA (mtDNA) which is inherited solely from the mother. Mitochondrial DNA is a small circular chromosome found inside mitochondria. The main function of mitochondrial DNA is to provide instructions for production of enzymes and membrane structures involved in oxidative phosphorylation. In senescent cells, due to compensation mechanisms, mtDNA quantity is increased and can be visualized using fluorescent labels and measured as density of fluorescent puncta in cell body (mtDNA content) or percentage of cell volume occupied by mtDNA (mitochondrial mass).

Fibronectins are glycoproteins that connect cells with collagen fibers in the extracellular matrix, allowing cells to move through the matrix. Fibronectin has profound effects on would healing, including the formation of proper substratum for migration and growth of cells during the development and organization of granulation tissue, as well as remodeling and synthesis of connective tissue matrix. Accordingly, fibronectin also positively affects the skin texture, in particular the firmness of the skin.

Fibronectins are secreted by cells in an unfolded, inactive form. Binding to integrins triggers conformation changes in fibronectin molecules, allowing them to form dimers. Those dimers bind collagen and cell-surface integrins, causing a reorganization of the cell's cytoskeleton to facilitate cell movement (Freeman and Hamilton; Pearson Prentice Hall, 2005).

Collagen Typ III is an important structural component of the skin and thus important to maintain skin texture, in particular the firmness of the skin. In addition, it is also involved in blood clotting. In the later stages of wound healing, the rebuilding of the extracellular matrix is an important process, in which collagen Typ III plays a role.

In a preferred embodiment of the cosmetic use described above, the β-L-aspartyl-L-arginine provides one or more effect(s) selected from the group consisting of

a) protection of senescent skin cells against mitochondrial reactive oxygen species (ROS)-induced damages;

(b) protection against UV-A induced reactive oxygen species (ROS) in dermis;

(c) protection against UV-induced oxidation, in particular protein carbonylation, in senescent skin cells;

(d) reduction of mitochondrial reactive oxygen species (ROS) in senescent skin cells;

(e) reduction of mitochondrial DNA (mtDNA) in senescent skin cells;

(f) reduction of cell size in senescent skin cells;

(g) reduction of the amount of senescent skin cells;

(h) reduction of nucleus to cell size ratio in senescent skin cells;

(i) stimulation of collagen Type III production in senescent skin cells; and

(j) stimulation of fibronectin production in senescent skin cells.

Preferably, the above effects (a) to (j) are obtained in senescent fibroblasts. In a preferred embodiment of the cosmetic use described above, the senescent skin cells are senescent fibroblasts.

The effects (a) to (j) have been demonstrated in the examples below to be pronounced in senescent cells and thus the dipeptide allows effective cosmetic treatment specifically of mature skin.

In one embodiment of the cosmetic use described above, the β-L-aspartyl-L-arginine is applied topically to the skin.

It has been demonstrated that the above described effects are obtained by topical application of β-L-aspartyl-L-arginine on the skin. The dipeptide can be applied e.g. in a cosmetic formulation such as a crème, a spray, a lotion, an ointment or a gel comprising one or more cosmetically acceptable carriers and excipients. A skilled person is aware how to prepare such cosmetic preparations.

In a further embodiment of the cosmetic use described above, the β-L-aspartyl-L-arginine is used in a cosmetic composition comprising 0.001 to 2 wt.-%, preferably 0.05 to 0.5 wt.-%, particularly preferably 0.1 to 0.3 wt.-% β-L-aspartyl-L-arginine, in each case with respect to the total weight of the composition.

The above specified dosages are typically used in cosmetic formulations and provide the desired effect(s). It is advantageously not necessary to use higher amounts of the dipeptide and thus increase production cost.

The present invention also relates to a cosmetic method for the treatment of mature skin comprising the step:

(i) applying β-L-Aspartyl-L-Arginine topically on mature skin.

As explained above and demonstrated in the examples below, application of the dipeptide on mature skin provides a number of beneficial effects.

In one embodiment of the cosmetic method described above, the β-L-aspartyl-L-arginine provides an antioxidative effect and/or improves mitochondrial function in senescent skin cells, in particular senescent fibroblasts, and/or restores firmness of the skin, in particular, the β-L-aspartyl-L-arginine provides one or more effect(s) selected from the group consisting of

a) protection of senescent skin cells against mitochondrial reactive oxygen species (ROS)-induced damages;

(b) protection against UV-A induced reactive oxygen species (ROS) in dermis;

(c) protection against UV-induced oxidation, in particular protein carbonylation, in senescent skin cells;

(d) reduction of mitochondrial reactive oxygen species (ROS) in senescent skin cells;

(e) reduction of mitochondrial DNA (mtDNA) in senescent skin cells; (f) reduction of cell size in senescent skin cells;

(g) reduction of the amount of senescent skin cells;

(h) reduction of nucleus to cell size ratio in senescent skin cells;

(i) stimulation of collagen Type III production in senescent skin cells; and

(j) stimulation of fibronectin production in senescent skin cells.

Preferably, the above effects (a) to (j) are obtained in senescent fibroblasts. In a preferred embodiment of the cosmetic method described above, the senescent skin cells are senescent fibroblasts.

Since it has been demonstrated that the above described effects are pronounced in mature skin comprising a significant amount of senescent cells, in the cosmetic method described above, the mature skin is the skin of an individual, which is at least 40, at least 45, at least 50, at least 55, at least 60, at least 65, or at least 70 years old.

In a preferred embodiment of the cosmetic method described above, the β-L-aspartyl-L-arginine is applied in a cosmetic composition comprising 0.001 to 2 wt.-%, preferably 0.05 to 0.5 wt.-%, particularly preferably 0.1 to 0.3 wt.-% β-L-aspartyl-L-arginine, in each case with respect to the total weight of the composition.

The present invention also relates to β-L-aspartyl-L-arginine for use in a therapeutic method for the treatment of damaged mature skin.

Damaged skin in this context means in particular that the skin has been injured, e.g. cut, punctured, scratched or burned. The function of the damaged skin therefore has been compromised and requires a healing process to be restored. In particular, in mature skin, which comprises a significant amount of senescent cells, the healing process is usually slowed down compared to younger skin.

Preferably, the damaged mature skin is the skin of an individual, which is at least 40, at least 45, at least 50, at least 55, at least 60, at least 65 or at least 70 years old.

As demonstrated in the examples below, application of β-L-aspartyl-L-arginine to the skin, stimulates fibronectin and collagen Type III production and thus accelerates wound healing. In a preferred embodiment, the β-L-aspartyl-L-arginine therefore promotes wound healing.

Short description of the figures:

In the FIGS. β-L-aspartyl-L-arginine is designated by the batch numbers BIO 4618, BIO 4619 and BIO 4620.

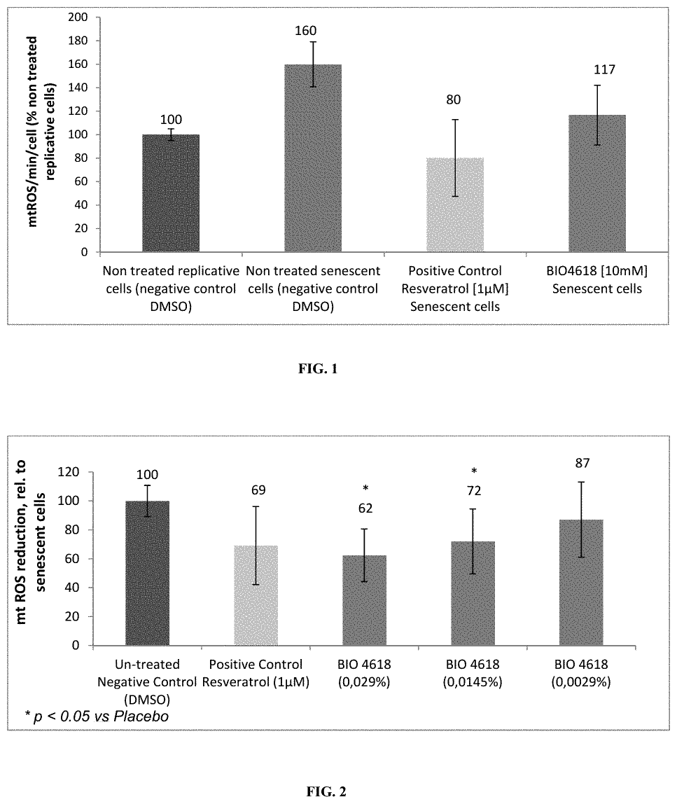

FIG. 1 shows the reduction of mitochondrial ROS in senescent fibroblast by β-L-aspartyl-L-arginine as measured in the in vitro study described in example 1.

FIG. 2 shows the dosage dependence of the reduction of mitochondrial ROS in senescent fibroblast by β-L-aspartyl-L-arginine as measured in the in vitro study described in example 1.

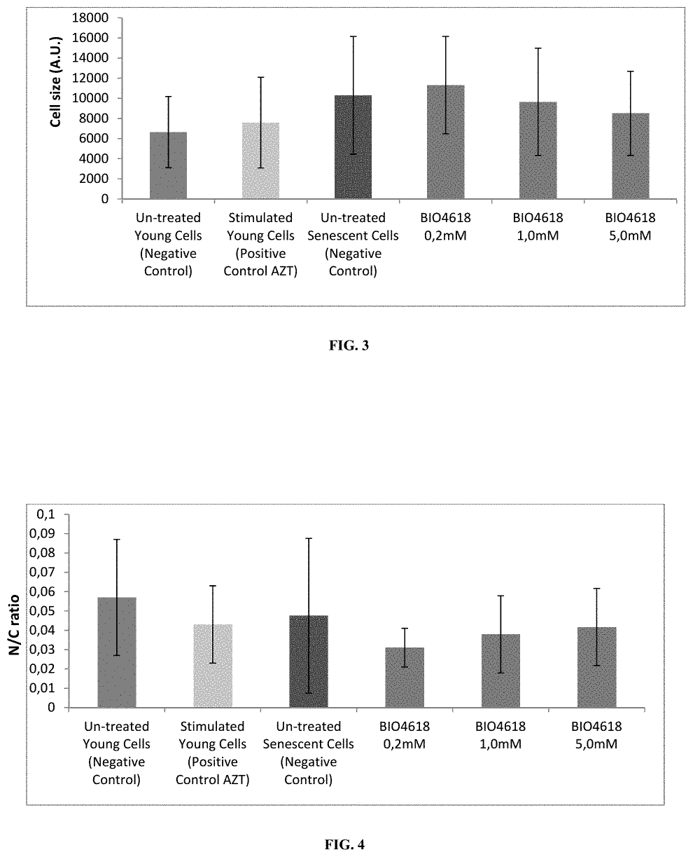

FIG. 3 shows the reduction of cell size in senescent fibroblast by β-L-aspartyl-L-arginine as measured in the in vitro study described in example 2.

FIG. 4 shows the reduction of the nucleus to cell size (N/C) ratio in senescent fibroblasts by β-L-aspartyl-L-arginine as measured in the in vitro study described in example 3.

FIG. 5 shows the reduction of mitochondrial DNA (mtDNA) in senescent fibroblasts by β-L-aspartyl-L-arginine as measured in the in vitro study described in example 4.

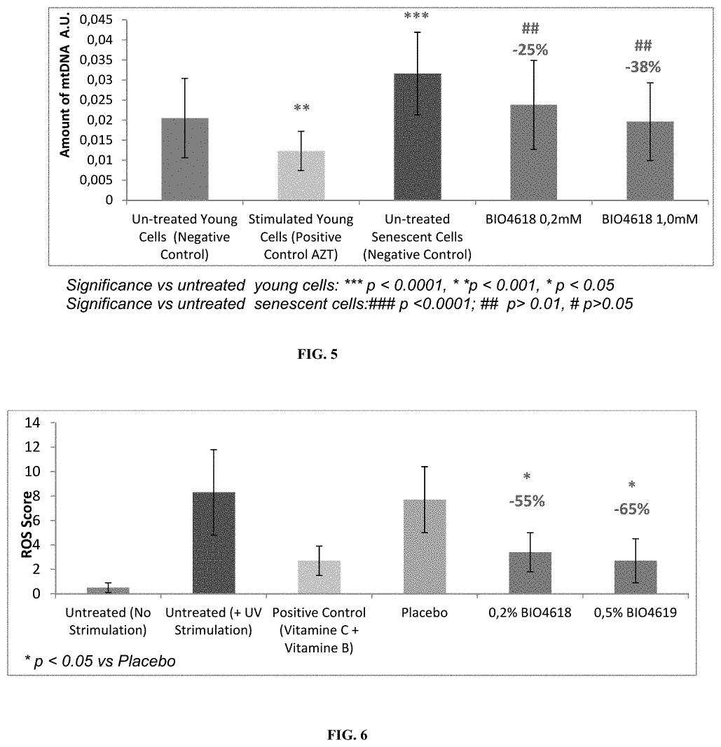

FIG. 6 shows the reduction of the ROS score by β-L-aspartyl-L-arginine after UVA irradiation of human skin explants as measured in the ex vivo study described in example 5.

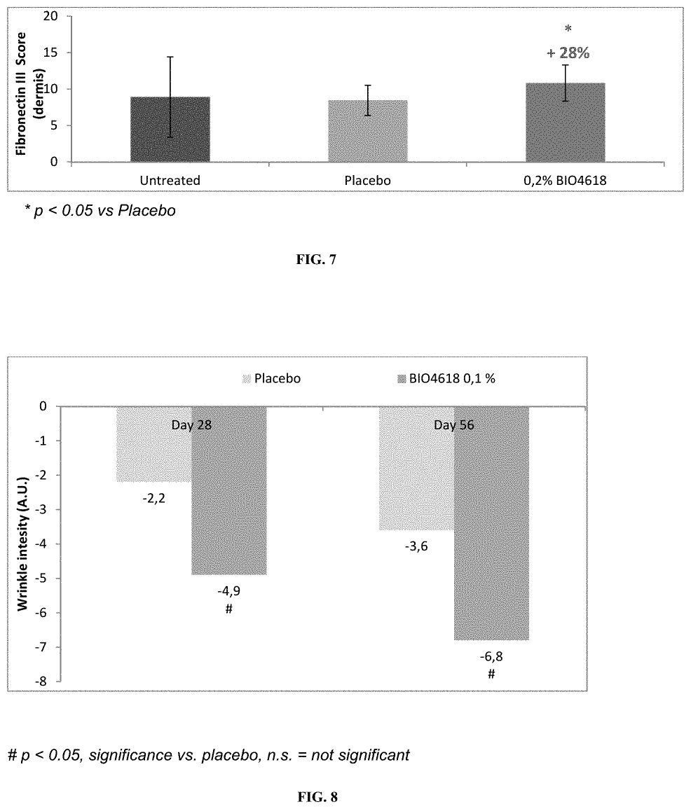

FIG. 7 shows the stimulation of the production of fibronectin proteins by β-L-aspartyl-L-arginine in the dermis as measured in the ex vivo study described in example 6.

FIG. 8 shows the reduction of wrinkle intensity by β-L-aspartyl-L-arginine as measured in the clinical study described in example 7.

FIG. 9 shows the protection of human skin explants against carbonylation of proteins by β-L-aspartyl-L-arginine as measured in the ex vivo study described in example 8.

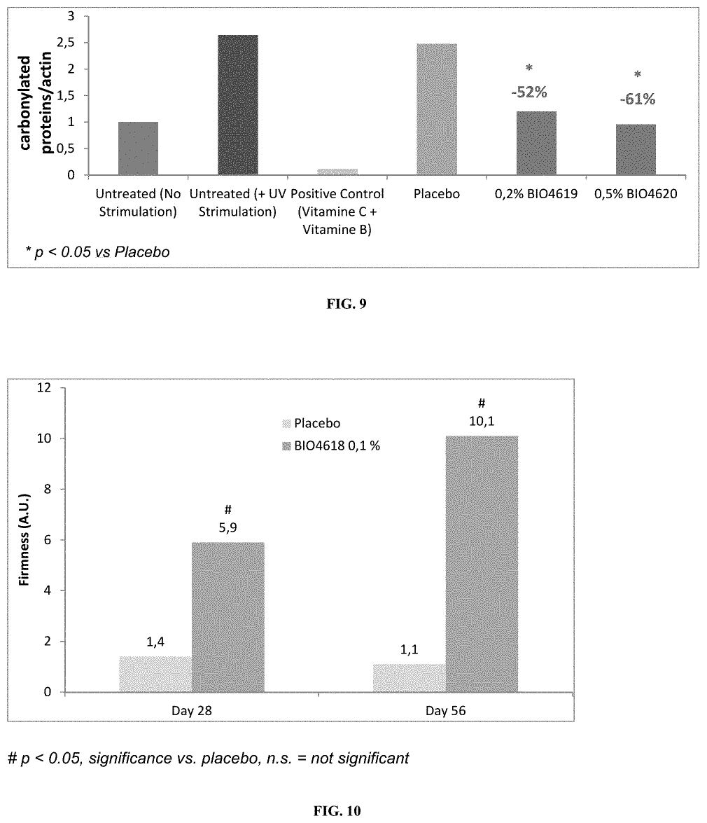

FIG. 10 shows the improvement of skin firmness by β-L-aspartyl-L-arginine as measured in the clinical study described in example 9.

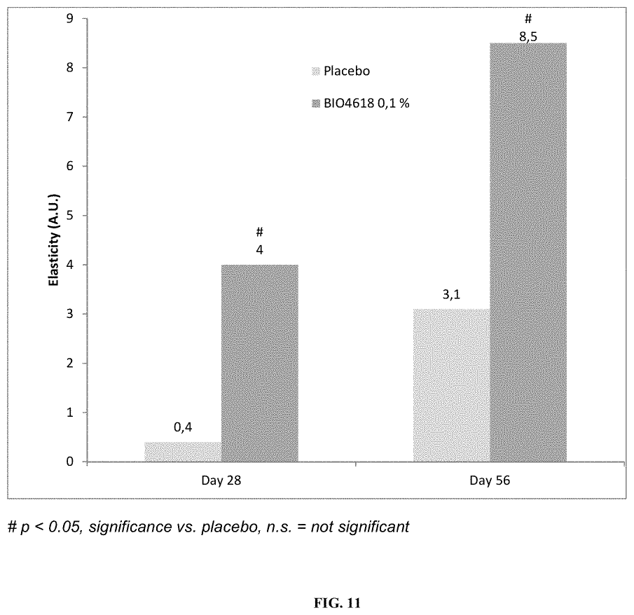

FIG. 11 shows the improvement of skin elasticity by β-L-aspartyl-L-arginine as measured in the clinical study described in example 9.

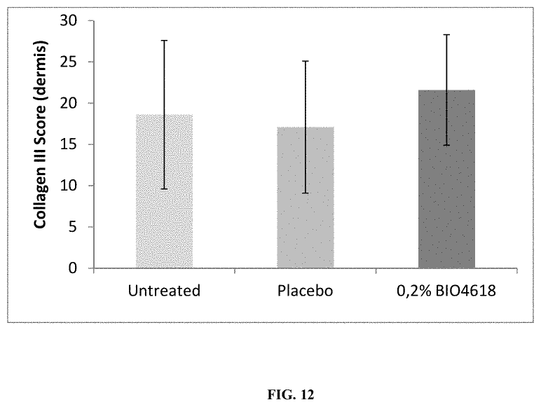

FIG. 12 shows the stimulation of collagen Type III by β-L-aspartyl-L-arginine compared to untreated skin and a placebo.

### EXAMPLE 1

### Reduction of Mitochondrial ROS in Senescent Fibroblasts (In Vitro Study)

Using a fluorescent dye, the antioxidative potential of β-L-aspartyl-L-arginine with respect to mitochondrial reactive oxygen species (ROS) was quantified. Senescent cells were incubated with β-L-aspartyl-L-arginine for 24 hours. Subsequently, the treated and non-treated senescent cells as well as the treated and non-treated non-senescent cells were incubated with the fluorescent dye for 30 minutes at 37° C. After washing, a portion of the non-senescent cells was treated with the oxidative reference substance H₂O₂ (100 μM). The mitochondrial ROS production was measured immediately every 5 mins over a period of 60 minutes. A microtiter plate reader (Varioskan-Thermo) was used as measuring device. During the kinetic measurement, the cells were further incubated at 37° C. Using fluorimetry, the antioxidative activity was measured in parallel with the viability of the cell. The experiments comprised a blank as well as a negative and a positive control. The positive control consists of non-senescent cells, which were only treated with the vehicle (DMSO). These cells were compared to non-treated senescent cells (negative control). Senescent cells treated with resveratrol were used as reference for the comparison of the activity of β-L-aspartyl-L-arginine in the decrease of endogenous production of mitochondrial ROS in senescent cells. β-L-aspartyl-L-arginine was measured in a 4-fold measurement. The effects of β-L-aspartyl-L-arginine were simultaneously compared to the negative and the positive control.

As can be inferred from FIG. 1, with respect to non-treated senescent cells, the cells treated with 10 μM β-L-aspartyl-L-arginine show a significant reduction of mitochondrial ROS (mtROS), which is comparable to the effect achieved with resveratrol. Resveratrol is known to be able to reduce ROS and is used as a positive control. The values are shown with respect to non-treated replicative cells, which represent 100%.

In FIG. 2, it is shown that there is a dosage dependence of the mtROS reduction effect. Increasing amounts of β-L-aspartyl-L-arginine show a larger reduction. In this diagram, the values are shown with respect to the untreated senescent cells used as negative control representing 100%.

### EXAMPLE 2

### Reduction of Cell Size in Senescent Fibroblasts (In Vitro Study)

Senescent cells were incubated with β-L-aspartyl-L-arginine. Cell size and nuclear size were measured while cell density reflects the viability. The reference compound AZT (azidothymidine), a gamma polymerase inhibitor reduces mtDNA amount. See example 4 for the details of experimental procedure.

FIG. 3 shows that amounts of more than 1.0 mM β-L-aspartyl-L-arginine are able to reduce cell size (given in arbitrary units (A.U.)) of senescent fibroblasts compared to untreated senescent fibroblasts. By treatment with β-L-aspartyl-L-arginine, the cells size of senescent fibroblasts can be reduced so that it is comparable to that of young replicative fibroblasts.

### EXAMPLE 3

### Reduction of Nucleus to Cell Size (N/C) Ratio in Senescent Fibroblasts (In Vitro Study)

Senescent cells were incubated with β-L-aspartyl-L-arginine. Cell size and nuclear size were measured while cell density reflects the viability. The reference compound AZT (azidothymidine), a gamma polymerase inhibitor reduces mtDNA amount. See example 4 for the details of experimental procedure.

As can be inferred from FIG. 4, in the presence of β-L-aspartyl-L-arginine, the nucleus to cell size ratio in senescent fibroblasts is reduced compared to untreated senescent fibroblasts to reach the nucleus to cell size ratio of replicative young cells.

### EXAMPLE 4

### Reduction of Mitochondrial DNA (mtDNA) in Senescent Fibroblasts (In Vitro Study)

Disruptions of the nuclear and mitochondrial DNA were studied, analyzed by detecting nucleus fragments, the mitochondrial DNA content, the mitochondrial mass and the average cell density, the latter as representation of viability.

The cells were treated with β-L-aspartyl-L-arginine for 5 days. The cell images were taken after incubation. In order to label the nuclear and mitochondrial DNA, the cells were incubated with a fluorescent dye for 30 minutes. After removal of the dye, the images were taken with an epifluorescence microscope. Image analysis was performed in a 3-fold determination. Sampling 10 pictures per experimental condition or repetition, respectively.

Data collection: The images were taken with a Zeiss axioplan microscope. Image analysis was performed with a software developed by ICDD.

Data interpretation: The following data was collected: average cell size, mtDNA content, mitochondrial density, mitochondrial biogenesis, mitochondrial mass and cell nucelus size. The average cell number per field was used as control of cell viability for each experimental condition. The effect of active substances was also given as protective effect in comparison to non-treated cells. The effect of the positive control was included for comparison. FIG. 5 shows that the mtDNA in senescent cells is reduced by β-L-aspartyl-L-arginine to an amount comparable to young replicative cells.

FIG. 5 shows that the mtDNA in senescent cells is reduced by β-L-aspartyl-L-arginine 25 to an amount comparable to young replicative cells. The reference compound AZT (azidothymidine), a gamma polymerase inhibitor reduces mtDNA amount.

### EXAMPLE 5

### Reduction of ROS Score After UVA Irradiation on Human Skin Explants (Ex Vivo Study)

Cultivation of skin models: The skin was cut into about 8×3 mm large pieces (diameter×layer thickness of the models) and cultivated until the planned end of the experiment. Per treatment, six models each were used. The skin models were cultivated as air-liquid-interface cultures in a perforated stainless steel ring with contact to the culture medium (modified Williams 'E Medium) until the desired endpoint.

Treatment: Test substances and controls were applied topically. To this end, the skin models were carefully cleaned with a cotton pad. Subsequently, 4 μL of each test substance or the control, respectively, were applied to the models and covered with a membrane filter (diameter=6 mm).

The skin models were incubated for 18 h with test substance and positive control, respectively. Subsequently, the detection reagent (e.g. DCFH-DA) was added to the culture medium analog to the description of Marionnet et al. (Plos One, 9, 2014) After another 30 minutes, the DCFH-DA reagent was removed and the oxidative stimulation was performed (e.g UVA). Dichlorofluorescein diacetat reacts with ROS to form a fluorescent product.

UV-irradiation: The used sun simulator was a BIO-SUN-system from the company Vilber Lourmat. The system is based on a programmable microprocessor, which controls the UV-irradiation. The UV-light emission is constantly monitored and the system stops automatically when the pre-set energy quantity is reached. The irradiation cycles are therefore always reproducible—independent on the intensity loss of the UV lamp. In order to determine the UV-irradiation dose, the “biologically effective dose (BED)” was used, which is described in Del Bino et al. (Pigment Cell Research, 19, 2006).

Determination of ROS: At the end of the experiment, the skin models were harvested, cryoconserved and cut using a cryostat for the subsequent image capture and analysis. The fluorescent analysis was performed in the area of the dermis. The area selected for the analysis ran from the upper part of the dermis—following the basal layer—to the lower part of the dermis, while it was made sure that irregular structures such as blood vessels, sweat glands or hair follicles were avoided.

Of each skin model, two samples were used for image capture with the fluorescence analysis. Each picture was analyzed by determining the fluorescence using Image-J analysis software (NIH, USA). The measured values were normalized over the size of the selected surface.

Statistics: For each treatment of the skin models, the averages of the quantitative data was determined. For calculating the variations—standard deviation and standard error of the average (SEM)—the raw data was used. Differences between the treated skin models were calculated with one-way ANOVA with permutation test and subsequent Turkey and T-Test with permutation.

As can be inferred from FIG. 6, β-L-aspartyl-L-arginine is a potent UV-induced ROS scavenger in the dermis and significantly reduces the ROS score of UV irradiated skin in comparison to UV irradiated skin, which was not treated with β-L-aspartyl-L-arginine. Therefore, β-L-aspartyl-L-arginine provides a strong protection from UVA induced ROS damages in the dermis.

### EXAMPLE 6

### Stimulation of Fibronectin Proteins in the Dermis (Ex Vivo Study)

Collagen I, Collagen III, MMP1, Elastin, Fibrillin, Fibronectin

4 μl of β-L-aspartyl-L-arginine were applied on the skin every day during 6 days of treatment at 37° C., 5% CO₂ and 100% humidity. 12 skin cuts were immunohistochemically stained with selected antibodies. The papillary dermis was selected for the analysis because it is the part of the dermis, which changes the most in reaction to a previous treatment.

The area to be analyzed was carefully selected while it was made sure that irregular structures such as blood vessels, sweat glands or hair follicles were avoided.

For the assessment of the results, both, the color intensity and the color distribution were measured using Image-J analysis software (NIH, USA)—thus a semi-quantitative assessment was possible.

FIG. 7 shows that β-L-aspartyl-L-arginine is able to stimulate the fibronectin production in the dermis compared to untreated skin. It is therefore able to support skin renewal and wound healing.

FIG. 12 shows that β-L-aspartyl-L-arginine is able to stimulate the collagen Typ III production compared to untreated skin. It is therefore able to support skin renewal and wound healing.

### EXAMPLE 7

### Reduction of Wrinkle Intensity (Clinical Study)

The study was conducted with 26 female volunteers of age 53 +/−3 years. The application was done twice daily for two months. On one side of the face, the product is applied, on the other side the placebo.

Imaging: The evaluation is based on high-resolution digital photography under controlled conditions. Pictures were taken at the start of the study, after 28 days and after 56 days of product application.

The subjects were positioned in front of the optical arrangement and the face was photographed as portrait or close-up. The persons were photographed seated at controlled lighting. The conditions were kept constant during all photographs to ensure comparability.

Reduction of wrinkles and lines: The assessment of wrinkles and lines was done in a limited area around the eye of the subject. The intensity of the wrinkles and lines was measured as parameter R. The larger the value for R, the more pronounced are the wrinkles and lines. The relative reduction of wrinkles is calculated according to the following formula:

R%=100(Rᵢ−RF)/Rᵢ(R=wrinkle intensity, i=initial; f=final)

Data analysis and interpretation: For data analysis the following software packages were used:

Microsoft® Office Excel 2010 (Mircrosoft Corp., EUA, 2010)

GraphPad™ Prism® 6.00 (GraphPad Software, San Diego Calif. USA)

It can be inferred from FIG. 8, that β-L-aspartyl-L-arginine visibly smoothes wrinkles and fine lines on crow feet after only four weeks of twice daily use of 0.1%. After eight weeks, 85% of volunteers have observed a significant 6.8% reduction of wrinkle intensity compared to a placebo.

### EXAMPLE 8

### Protection Against Carbonylation of Proteins (Ex Vivo Study)

Detection of carbonylation: After removing the fatty and connective tissue, the frozen skin samples are weighed and cut. The skin pieces of the tested samples are combined and homogenized in a cold extraction buffer. Cell lysates are collected by centrifugation.

The measurement of the carbonylated protein is performed with the “OxyBlot Protein_Oxidation Kit by Millipore”, (#S7150) according to the protocol of the producer. Only the protein bands of a size between 50 kDa and 150 kDa were taken into account for the calculation. The obtained amount of carbonylated protein is normalized with the actin band (which is constant) and given with respect to the non-treated sample.

As can be inferred from FIG. 9, β-L-aspartyl-L-arginine is a potent antioxidant at protein level and is able to prevent the formation of carbonylated proteins in an ex vivo model comparing untreated UV stimulated skin and UV stimulated skin treated with 0.2% and 0.5% β-L-aspartyl-L-arginine. The positive control was treated with vitamin C and vitamin B, which are known to provide an antioxidative effect.

### EXAMPLE 9

### Improvement of Skin Firmness and Elasticity (Clinical Study)

The study was conducted with 26 female volunteers of age 53 +/−3 years. The application was done twice daily for two months. On one side of the face, the product is applied, on the other side the placebo.

The method of measurement relies on suction. A vacuum is created in the gauge head of the cutometer, which sucks the skin into the measuring device. An optical arrangement, consisting of a light source and a light detector, measures the light intensity, which enters dependent on how much skin is sucked in. The resulting parameters are elasticity and skin firmness.

Skin firmness represents the resistance, which the skin creates against the suction by the vacuum. Elasticity, is the time, which the skin needs to return to its original state.

FIG. 10 shows how β-L-aspartyl-L-arginine visibly improves skin firmness after only four weeks of twice daily use of 0.1%. After eight weeks, 92% of volunteers observed a significant enhancement of skin firmness (+10.1%) compared to the placebo.

FIG. 11 shows how β-L-aspartyl-L-arginine visibly improves skin elasticity after only four weeks of twice daily use of 0.1%. After eight weeks, 100% of volunteers observed a significant enhancement of skin elasticity (+8.5%) compared to the placebo.

1-15. (canceled)

16. A method for treating inflammation of the skin comprising applying β-L-aspartyl-L-arginine to the skin of an individual.

17. The method of claim 16, wherein the method prevents or reduces secretion of one or more pro-inflammatory cytokines and/or and growth factors of skin cells.

18. The method of claim 17, wherein the pro-inflammatory cytokines and/or growth factors are chosen from interleukin 6 (IL-6), interleukin 8 (IL-8), vascular endothelial growth factor (VEGF), transforming growth factor (TGF), and combinations thereof

19. The method of claim 16, wherein the method treats and/or prevents chronic inflammation of the skin.

20. The method of claim 19, wherein the chronic inflammation is caused by the secretion of pro-inflammatory cytokines and/or growth factors by senescent skin cells.

21. The method of claim 16, wherein the method provides an antioxidative effect and/or improves mitochondrial function of mature skin in senescent skin cells.

22. The method of claim 21, wherein the method restores firmness to the skin.

23. The method of claim 21, wherein the individual is at least 40 years old.

24. The method of claim 21, wherein the β-L-aspartyl-L-arginine provides one or more effect(s) selected from:
(a) protection of the senescent skin cells against mitochondrial reactive oxygen species (ROS)-induced damages;
(b) protection against UV-A induced reactive oxygen species (ROS) in dermis;
(c) protection against UV-induced oxidation;
(d) reduction of mitochondrial reactive oxygen species (ROS) in the senescent skin cells;
(e) reduction of mitochondrial DNA (mtDNA) in the senescent skin cells;
reduction of cell size in the senescent skin cells;
(g) reduction of a number of the senescent skin cells;
(h) reduction of a nucleus to cell size ratio in the senescent skin cells;
stimulation of collagen Type III production in the senescent skin cells; and
stimulation of fibronectin production in the senescent skin cells.

25. The method of claim 21, wherein the β-L-aspartyl-L-arginine is applied topically to the skin.

26. The method of claim 21, wherein the method comprises applying to the skin of the individual a composition comprising:
0.001 to 2 wt.-% of the β-L-aspartyl-L-arginine, based on a total weight of the composition.

27. The method of claim 21, wherein the mature skin is damaged.

28. The method of claim 26, wherein the individual is at least 40 years old.

29. The method of claim 16, wherein the β-L-Aspartyl-L-Arginine promotes wound healing.

30. The method of claim 21, wherein the β-L-Aspartyl-L-Arginine promotes wound healing.

31. The method of claim 16, wherein the individual is at least 50 years old.

32. The method of claim 21, wherein the individual is at least 50 years old.

33. The method of claim 16, wherein the individual is an individual suffering from chronic inflammation.

34. The method of claim 21, wherein the individual is an individual suffering age-related photo-aging.

35. The method of claim 16, wherein the individual is in need of treatment for inflammation of the skin.
